(19) United States
(12) Patent Application Publication
(10) Pub. No.: US 2019/0257814 A1
(43) Pub. Date: Aug. 22, 2019

(54) Smart Rapid Result Diagnostic System

(71) Applicant: Milo IP Holder LLC, Fort Myers, FL (US)

(72) Inventor: Lawrence David Leigh, Fort Myers, FL (US)

(73) Assignee: Milo IP Holder LLC, Fort Myers, FL (US)

(21) Appl. No.: 15/902,701

(22) Filed: Feb. 22, 2018

(51) Int. Cl. G01N 33/487; H04L 29/08

(52) U.S. Cl. CPC G01N 33/48792; H04L 29/08558; G01N 33/48771

(57) ABSTRACT

A smart rapid result diagnostic system distributed on one or more servers and operably coupled to one or more client computing devices or one or more clinician computing devices by a public network which supports a smart rapid result diagnostic application accessible on-line or downloadable by the client computing devices to establish off-line connection with one or more assay devices operable to detect an analyte in a sample and provide locked or unlocked access to the assay results based on one or more references correlated with one or more assay tables in an assay table library.

### I. FIELD OF INVENTION

A smart rapid result diagnostic system distributed on one or more servers and operably coupled to one or more client computing devices or one or more clinician computing devices by a public network which supports a smart rapid result diagnostic application accessible on-line or downloadable by the client computing devices to establish off-line connection with one or more assay devices operable to detect an analyte in a sample and provide locked or unlocked access to the assay results based on one or more references correlated with one or more assay tables in an assay table library.

### II. SUMMARY OF THE INVENTION

A broad object of a particular embodiments of the invention can be to provide a computer implemented application program distributed on one or more servers and operably coupled to one or more client computing devices or one or more clinician computing devices by a public network accessible on-line or downloadable by the client computing devices to establish off-line connection with one or more assay devices operable to detect an analyte in a sample and provide locked or unlocked access by the client computing device to the assay results of the assay device.

A broad object of particular embodiments of the invention can be to provide an assay device which receives an assay material and generates an assay result indicative of a presence or an amount of an analyte bound to the assay material and which communicatively connects to a client computing device to transmit assay results accessible by the client device based upon correlation of the assay identification codes with one or more client identification references, coordinate location references, governmental or agency regulatory references, or clinician permission references.

A broad object of particular embodiments of the invention can be to provide client computing device having a client processor communicatively coupled with a non-transitory computer readable media containing a smart rapid result diagnostic application program executable to communicatively connect the client computing device to an assay device which generates an assay result indicative of a presence or an amount of an analyte bound to the assay material and receives assay results from the assay device and based upon correlation of one or more client identification references, governmental or agency regulatory references, coordinate location references, or clinician permission references provides unlocked access by the client device to the assay results.

Naturally, further objects of the invention are disclosed throughout other areas of the specification, drawings, photographs, and claims.

### III. BRIEF DESCRIPTION OF THE DRAWINGS

FIG. 1 is an illustration of a particular embodiment of an assay device which receives an assay material within an assay reader.

FIG. 2A is a block diagram of an illustrative computer means, network means and computer-readable media which provides computer-executable instructions to provide an embodiment of a smart rapid result diagnostic system.

FIG. 2B is an illustration of a particular embodiment of a clinician computing device.

FIG. 2C is an illustration of a particular embodiment of a client computing device

FIG. 3 is a block flow diagram of a particular embodiment of a method of using the smart rapid result diagnostic system.

FIG. 4 is an illustrative example of a graphical user interface including sign up and login menus depicted on the display surface of a client computing device.

FIG. 5 is an illustrative example of a graphical user interface including a set up menu depicted on the display surface of a client computing device including an age status entry field.

FIG. 6 is an illustrative example of a graphical user interface including a set up menu depicted on the display surface of a client computing device including a gender status entry field.

FIG. 7 is an illustrative example of a graphical user interface including a set up menu depicted on the display surface of a client computing device including a weight status entry field.

FIG. 8 is an illustrative example of a graphical user interface including a set up menu depicted on the display surface of a client computing device including a height status entry field.

FIG. 9 is an illustrative example of a graphical user interface including a set up menu depicted on the display surface of a client computing device including a medicaments and food or drink entry field.

FIG. 10 is an illustrative example of a graphical user interface including a set up menu depicted on the display surface of a client computing device including a condition selection menu.

### IV. DETAILED DESCRIPTION

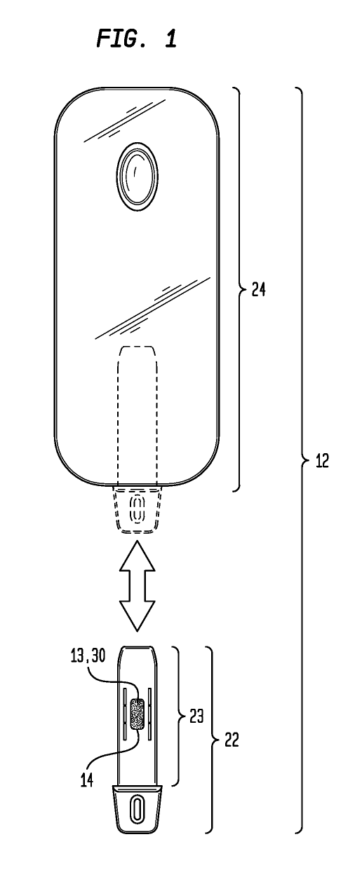

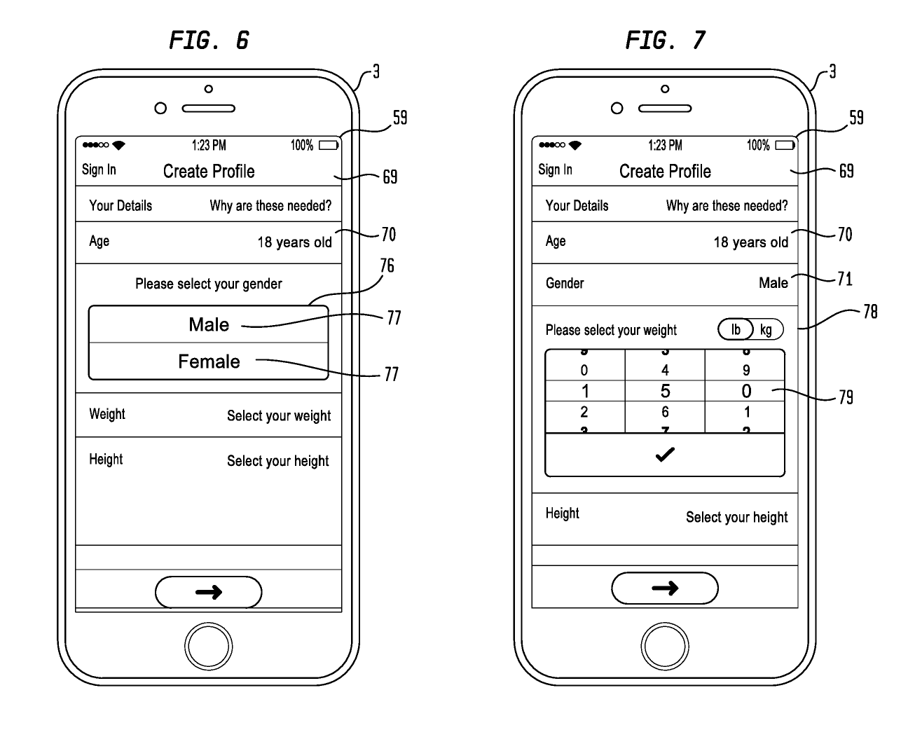

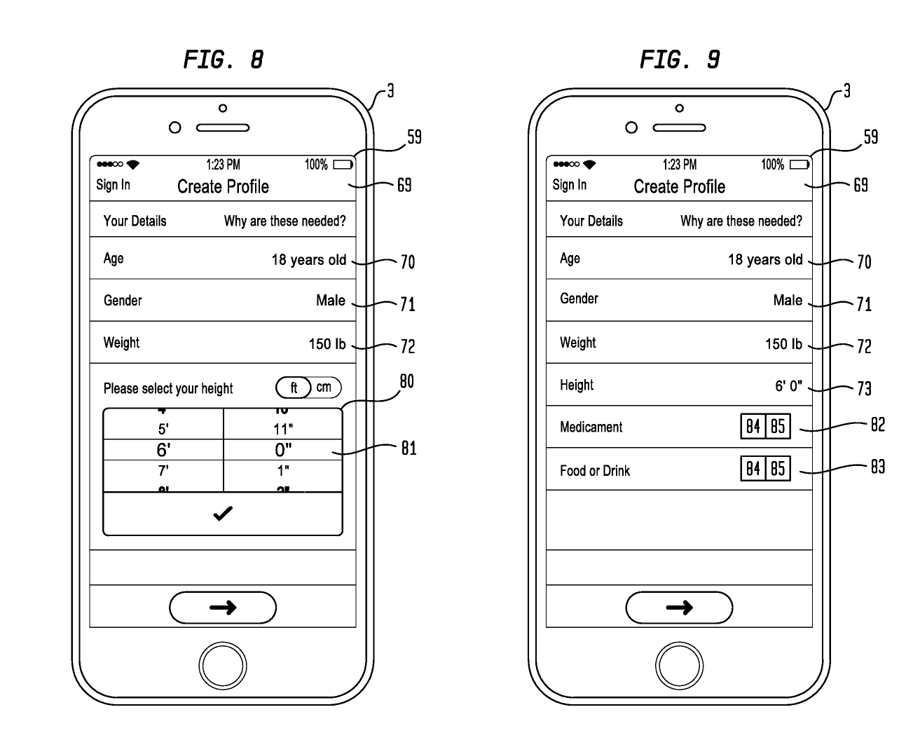

The System. Generally, referring to FIGS. 1 through 10, a smart rapid result diagnostic system (1) (also referred to as the “assay system (1)”) can be distributed on one or more servers (2) operably coupled to one or more client computing devices (3) and one or more clinician computing devices (4) by a public network (5), such as the Internet (6), a cellular-based wireless network(s) (7), or a local network (8) (individually or collectively the “network”). Each client computing device (3) and each clinician computing device (4) can further include a global positioning receiver (9) operably coupled to a global positioning system (“GPS”) (10). The client computing device (3) and the clinician computing device (4) can include as illustrative examples:

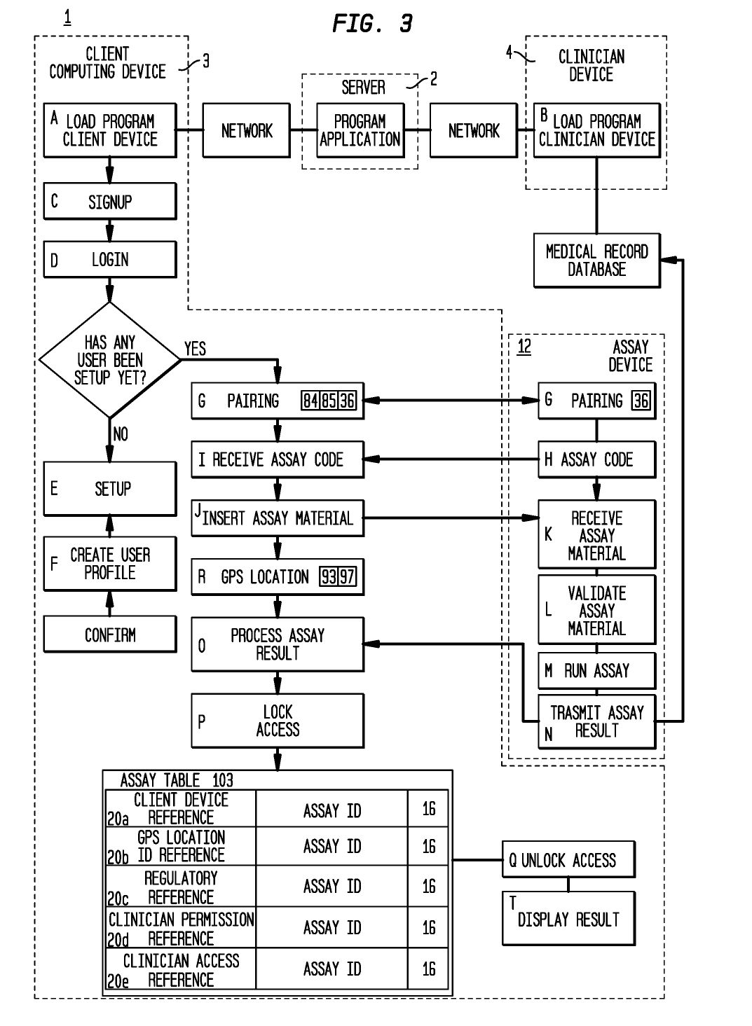

desktop computer devices, and mobile computer devices such as personal computers, slate computers, tablet or pad computers, cellular telephones, personal digital assistants, smartphones, programmable consumer electronics, or combinations thereof. The network (5) supports a smart rapid result diagnostic application (11) (also referred to as the “application program”) accessible by browser based on-line processing or downloadable by the client computing devices (3) to enable client computing devices (4) to establish off-line wired or wireless connection with one or more assay devices (12) operable to detect an analyte (13) in a sample (14) and to provide an assay data processing module (15) operable by the client computing device (3) or the clinician computing device (4) to receive an assay result (16) from the assay device (12) and to further determine if the assay result (16) can be accessed by the client computing device (3) (also referred to as “unlocked access (17)”) or cannot be accessed by the client computing device (3) (also referred to “locked access (18)”) based on one or more references (19). In the illustrative example described and shown in FIG. 3, the references (19) can be one or more of: client device identification reference (20a), global positioning references (20b), a government of regulatory reference (20c), a clinician permission references (20d), a clinician access reference (20e) or other references (19) correspondingly received or entered into the client computing device (3) or the clinician computing device (4).

The Assay Device. Now referring primarily to FIGS. 1 through 3, in particular embodiments, the assay device(s) (12) can be structured to practice self-screening or point of care procedures which can, but need not necessarily, meet over-the-counter regulatory standards in the

United States or comparable standards in foreign countries. In particular embodiments, the assay device (12) can be structured to practice only over-the-counter procedures or assays (for example, FDA Class I in vitro diagnostic device (or other medical devices). However, in particular embodiments, the invention may further encompass assay devices (12) structured to practice procedures or assays which may otherwise be delimited to research laboratories, analytical testing laboratories, by prescription, or otherwise fall outside of self-screening or point of care procedures (for example FDA Class II, III or IV in vitro diagnostic device (or other medical devices).

Typically, assay devices (12) include an assay material (22) (also referred to as a “test strip”) having an analyte capture region (23) capable of retaining or selectively binding an analyte (13) in a sample (12) and an assay material reader (24) into which the assay material (22) can be disposed or received to sense a presence or an amount of analyte (13) in the sample (14).

For the purposes of this invention, the term “sample (14)” means a liquid or solid material that can be applied to the assay material (22). A solid sample such as feces can, but need not necessarily, be dissolved in a suitable solvent before being applied to the assay material. Similarly, a liquid sample can, but need not necessarily, be treated with another liquid (such as water or an aqueous solution) to reduce its viscosity or increase its volume before being applied to the assay material (22). The sample (14) can be derived from any source. In particular embodiments, the source can, but need not necessarily, be a bodily fluid, such as one or more of: blood, serum, plasma, saliva, sputum, ocular lens liquid, sweat, urine, milk, ascites liquid, mucous, synovial liquid, peritoneal liquid, transdermal exudates, pharyngeal exudates, bronchoalveolar lavage, tracheal aspirations, cerebrospinal liquid, semen, cervical mucus, vaginal or urethral secretions, and amniotic liquid.

For the purposes of this invention, the term “analyte (13)” means any substance whose chemical constituents are being identified or measured. As illustrative examples an analyte (13) can include one or more of: therapeutic drugs, drugs of abuse, hormones, vitamins, proteins, antibodies, peptides, steroids, bacteria, fungi, viruses, parasites, antigens, allergens, T4, T3, digoxin, hCG, insulin, theophylline, luteinizing hormone, organisms causing or associated with various disease states, such as streptococcus pyogenes (group A), Herpes Simplex I and II, cytomegalovirus, chlamydiae, human bone alkaline phosphatase antigen (HBAPAg), human chorionic gonadotropin (hCG); human luteinizing hormone (hLH), human follicle stimulating hormone (hFSH), creatine phosphokinase MB isoenzyme; ferritin, carcinoembryonic antigen, prostate specific antigen; CA-549 breast cancer antigen, hepatitis B surface antigen, hepatitis B surface antibody, hepatitis B core antigen, hepatitis B core antibody, hepatitis A virus antibody, an antigen of human immunodeficiency virus HIV I, such as gp120, p66, p41, p31, p24 or p17, the p41 antigen of HIV II, and the respective anti-ligand (whether polyclonal or monoclonal antibody) to any one of the above ligands.

For the purposes of this invention the term “analyte capture region (23)” means the portion or region of the assay material (22) capable of selectively binding an analyte (13) in a sample (14). In particular embodiments, the sample (14) can, but need not necessarily be, contacted directly with the analyte capture region (23) where a reaction or binding event occurs to afford an assay result (16). In particular embodiments, the sample (14) can be applied to the assay material (22) and subsequently conveyed along the assay material (22), for example by capillary action, to the analyte capture region (23) where a reaction or binding event occurs to afford an assay result (16). As an illustrative example, the analyte (13) in the sample (14) can be conveyed to interact with a labelled specific binding reagent (also referred to as a “label”) to form a labelled complex (30). The label can comprise as illustrative examples: a metallic sol (such as gold), non-metallic elemental sol (such as selenium or carbon), dye sols, colored latex or polystyrene particles, or combinations thereof. The labelled complex (3) can comprise a colored particle. The labelled complex can be conveyed to and selectively bound to the analyte capture region (23), and thereby concentrated to indicate a presence or an amount of analyte (13) in the sample (14).

The assay device (12) can further include an assay material reader (24). The assay material (22) can be associated with, disposed on or inserted within the assay material reader (24). The assay material reader (24) can then operate a sensor (25) which generates an analyte measurement signal (26) indicative of a presence or an amount of the analyte (13) bound or selectively bound to said analyte capture region (23) of the assay material (22). In particular embodiments, the assay material (22) can be disposed or inserted to align the analyte capture region (23) in an assay material detection zone (27) of the assay material reader (24) and an analyte measurement sensor (25) can operate to generate an analyte measurement signal (26) indicative of a presence or an amount of the analyte (13) associated, bound or selectively bound to the analyte capture region (23) of the assay material (22). In particular embodiments, the assay material reader (24) can include a source of electromagnetic radiation (28) and one or more sensors (25) capable of detecting intensity of electromagnetic radiation (29). The source of electromagnetic radiation (28) and the sensors (25) can be positioned within the assay material reader (24) such that when the material assay reader (24) admits or receives the assay material (22) or test strip, the electromagnetic radiation (29) impacts the analyte (13) or labelled complex (30) upon the assay material (22).

In principle, any electromagnetic radiation (29) can be used to affect a fluorescence, a light absorption, or a light transmission measurement. In particular embodiments, the wavelength of the electromagnetic radiation (29) can be chosen at or near a wavelength which influences (results in luminescence of the analyte (13) or labeled complex (30)) or is influenced by (absorbed, by the analyte (13) or analyte complex (30)). In particular embodiments, the fluorescence emission of the analyte (13) or labelled complex (30) can be sensed by the sensor (25), such as a photosensor. In particular embodiments, the electromagnetic radiation (29) impacts the analyte (13) or labelled complex (30) upon the assay material (22) and the amount of reflected electromagnetic radiation (29) can be sensed by the sensor (25). In particular embodiments, the sensor (25) can be an analyte image capture element (25′) which generates an analyte measurement signal (26) in the form of analyte image pixel signals (26′) which vary based on luminance and color differences relating to the presence or amount of analyte (13) or analyte complex (30).

The assay reader (24) can further include a reader assay data processor (31) operable to receive the analyte measurement signal (26, 26′) indicative of the presence or the amount of the analyte (13) bound or selectively bound to said analyte capture region (23). The analyte measurement signal (26, 26′) can corresponding vary based on the presence or amount of the analyte (13) or analyte complex (30) bound to the analyte capture region (23). In particular embodiments, the reader assay data processor (31) can include an analog to digital convertor (21) to covert analog analyte measurement signals (26, 26′) into a digital signal (26″) The reader assay data processor (31) compare intensity of the analyte measurement signal (26, 26′, 26″) to one or more control thresholds (32). The reader assay data processor (31) can generate an assay result (16) based on comparison of the analyte measurement signal (26, 26′, 26″) to the one or more control thresholds (32). Typically, intensity of the analyte measurement signal (26) less than a control threshold (32) generates a negative result (33) while intensity of the analyte measurement signal (26) greater that the control threshold (32) generates a positive result (34) indicating the presence of the analyte (13, 30) or intensity of the analyte measurement signal (26) greater than a first control threshold (32′) but less than a second control threshold (32″) generates result (34) indicating the relative amount of the analyte (13, 30).

The assay reader (24) can further include a reader electronic data exchanger (35) operable to transmit assay material reader pairing information (36) to the client computing device (3). In particular embodiments, the assay device (12) can correspondingly include a radio frequency controller (37) which operates a radio frequency transmitter (38) to cause wireless connection or pairing of the assay device (12) with a client computing device (3) over a short-range radio frequency band (39) to carry a signal over all or a part of the communication path between the assay device (12) and a client computing device (3). The short-range frequency band (39) can include, as illustrative examples: BLUETOOTH® (40) which operates at frequencies of about 2402 MHz to about 2480 MHz or about 2400 MHz to about 243.5 MHz or WI-FI® (41) which operates at about 2.4 GHz or 5 GHz. In other particular embodiments, the assay device (12) can, but need not necessarily include, a tone generator (42) which generates tones (43) also referred to as an “audio beacon” that provides a signal over the communication path between the assay device (12) and a client computing device (3).

In particular embodiments, the reader electronic data exchanger (35) can further operate to transmit assay identification code (44)(shown in FIG. 3 as process (H)) from the assay material reader (12) to be received by the client computing device (3). The assay identification code (44) can be string of data which can be correspondingly associated with an assay material (22) to perform an assay (45) of a particular analyte (13) in a particular sample (14) (or a plurality of assay materials to perform a plurality of assays of a corresponding plurality of analytes). For example, the assay device (12) can transmit a string of data such as U12.B1.F1.xx1111 where U12 identifies the sample (14) as urine, and 1111 which identifies the batch number of the assay material (22) that has an intended uniform character or quality as it relates to the assay (45) of a particular analyte (13) or labeled complex (30). The assay device (12) can further operate to transmit assay device status data (46). For example, “B1” in the data string can indicate that the battery (47) has sufficient charge to run the assay (45). “F1” in the data string can indicate that the assay device has sufficient reagent fluid (48) to run the assay (45). The assay identification code (44) or the assay device status data (46) can be received by a client computing device (3).

In particular embodiments, the reader electronic data exchanger (35) can further operate to transmit an assay result (16) based on the comparison of the analyte measurement signal (26) to one or more control threshold(s) (32). The assay device (12) can send the assay result (16) in a string of data, such as, 1.2.3.4.5.6.7.8.9.10.11.12 where the data corresponds to the assay result (16) for one or more analytes (13) or labeled complexes (30) assayed. The data string can correspond to quantitative amounts of one or more analytes (13) or can correspond to binary determination of presence of one or more analytes (13). For example, 1 can correspond to a positive assay result (34) and 0 can correspond to a negative assay result (33).

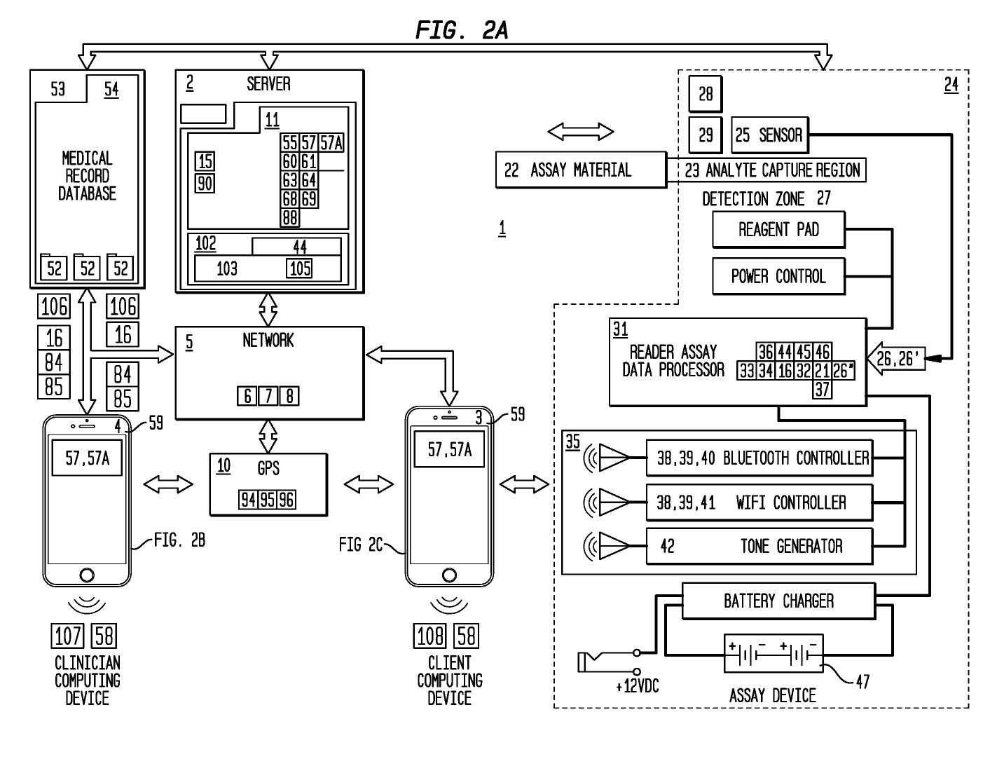

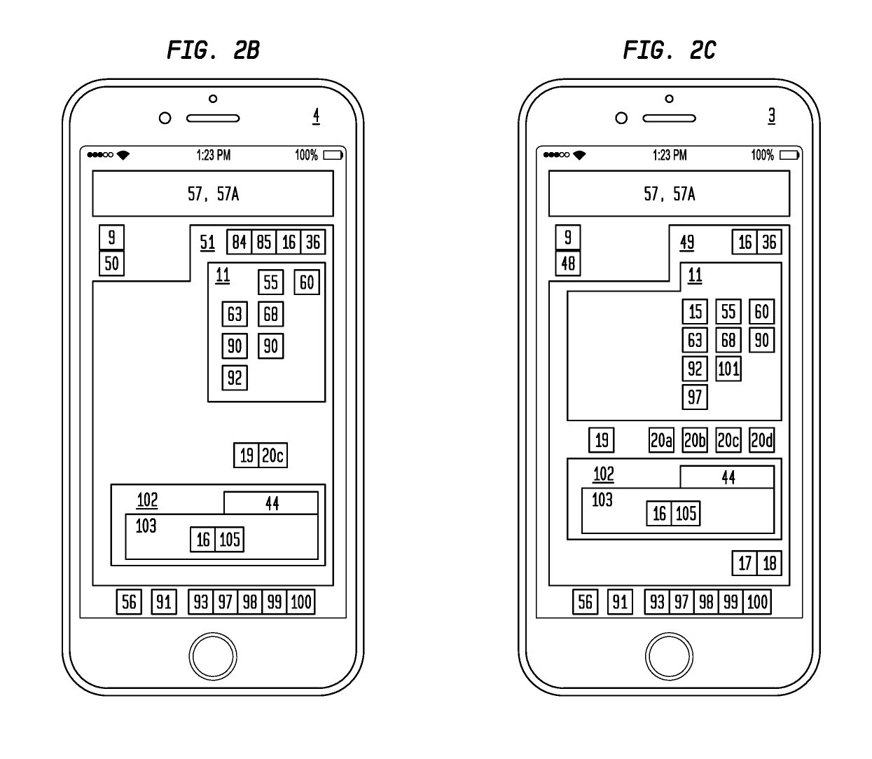

The Client Computing Device. Now referring primarily to FIGS. 2A, 2C and 3, a client computing device (3) can include a client device processor (48) communicatively coupled to a client device non-transitory computer readable media (49) containing computer executable instructions including in part the application program (11) to implement the functionalities of the client computing device (3) in the system (1). The client computing device (3) can as illustrative examples be: a desktop computer device or a mobile computer device, such as, personal computers, slate computers, tablet or pad computers, cellular telephones, personal digital assistants, smartphones, programmable consumer electronics, or combinations thereof. The application program (11) accessed or downloaded to the client computing device (3) can allow a client (50) access to the functionalities of the system (1) whether on-line or off-line depending on the application.

The Clinician Computing Device. Again, referring primarily to FIGS. 2A, 2B and 3, embodiments of the system (1) can, but need not necessarily, include a clinician computing device (4). The clinician device (4) can include a clinician device processor (50) communicatively coupled to a clinician device non-transitory computer readable media (51) containing computer executable instructions including in part the application program (11) to implement the functionalities of the clinician computing device (4) in the system (1). The clinician computing device (4) can as illustrative examples be: a desktop computer device or a mobile computer device, such as, personal computers, slate computers, tablet or pad computers, cellular telephones, personal digital assistants, smartphones, programmable consumer electronics, or combinations thereof. The application program (11) accessed or downloaded to the clinician computing device (4) can allow a clinician (107) access to the functionalities of the system (1) including access to the assay (45) test results (16) in electronic medical records (52) of a client (108).

The Electronic Medical Record. The term “electronic medical records (52)” refers the legal record of client (108) encounters and medical services received from one or more care delivery organizations (53). The electronic medical records (52) of a client (108) may be contained in one or a plurality of medical record databases (54). As an illustrative example, a care delivery organization (53) might include a hospital and a clinic owned by the same parent company and using a common format for electronic medical records (52) which are stored in the common medical record database (54). However, the care delivery organization (53) can be a plurality of unaffiliated health providers correspondingly using one or more unaffiliated medical record databases (54).

The Smart Rapid Result Diagnostic Application. Again, referring primarily to FIGS. 1 2A through 2C and 3, in particular embodiments, the application program (11) can be a smart rapid result diagnostic application program which can be accessed by or downloaded from one or more servers (2) to the client computing device (3)(as shown in the flow diagram of FIG. 3 as process (A)) or the clinician computing device (4)(as shown in the flow diagram of FIG. 3 as process (B)) to confer all of the functions of the application program (11) and the system (1) to the client computing device (3) or the clinician computing device (4).

In particular embodiments, the application program (11) can be executed to communicate with the server (2) over the network (5) to coordinate operation of the client computing device (3) with operation of the assay device (12) or a clinician computing device (4). However, this is not intended to preclude embodiments in which the application program (11) may be contained on and loaded to the client computing device (3), the clinician computing device (4) or the assay device (12) from one or more of: a computer disk, universal serial bus flash drive, or other computer readable media. While embodiments of the application program (11) are described in the general context of computer-executable instructions such as program modules which utilize routines, programs, objects, components, data structures, or the like, to perform particular functions or tasks or implement particular abstract data types, it is not intended that any embodiments be limited to a particular set of computer-executable instructions or protocols. Additionally, in particular embodiments, while particular functionalities of the application program (11) may be attributable to one of the assay device (12), the client computing device (3) or the clinician computing device (4); it is to be understood that embodiments may allow implementation of a function by more than one device, or the function may be coordinated between more than one device.

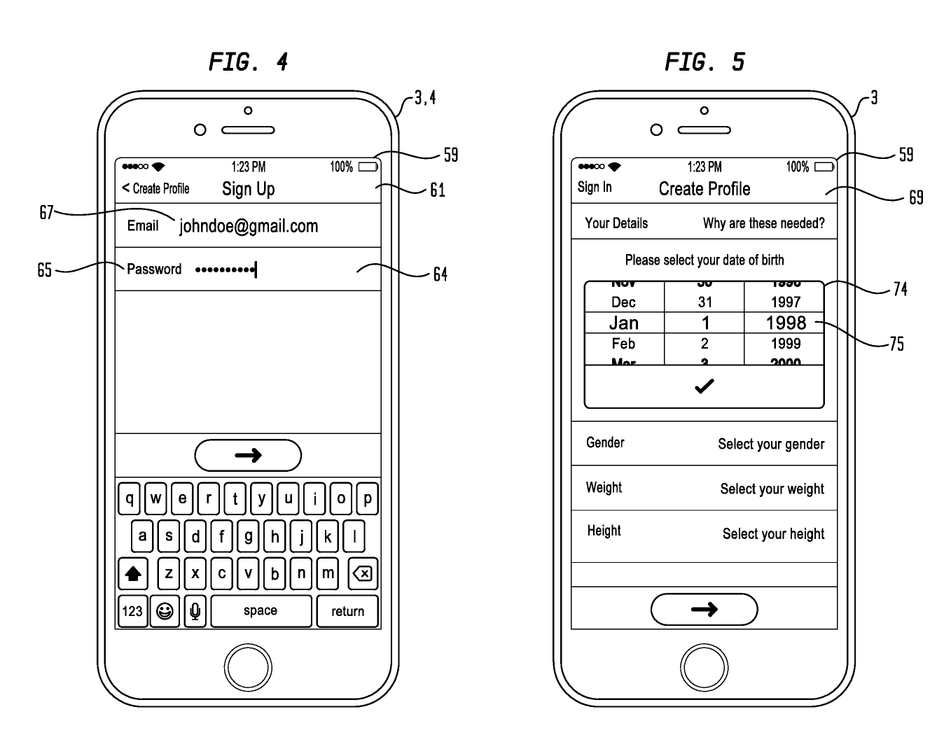

Now referring primarily to FIGS. 2A, 2B, and 2C each of the one or more client computing devices (3) or the clinician computing device (4) can, but need not necessarily, include an Internet browser (56) (also referred to as a “browser”), as illustrative examples: Microsoft's INTERNET EXPLORER®, GOOGLE CHROME®, MOZILLA®, FIREFOX®, which functions to download and render computing device content formatted in “hypertext markup language” (HTML). In this environment, the one or more servers (2) can contain the application program (11) including a graphical user interface module (55) which implements the most significant portions of one or more graphical user interface(s)(57) including one or more menus (57A) including a combination of text and symbols to represent options selectable by user command (58) to execute the functions of the application program (11). As to these embodiments, the one or more client computing devices (3) or the clinician computing device (4) can use the Internet browser (56) to depict the graphical user interface (57) including selectable menus (57A) and computing device content and to relay selected user commands (58) back to the one or more servers (2). The one or more servers (2) can respond by formatting additional menus (57A) for the respective graphical user interfaces (57) (as shown in the illustrative examples of FIGS. 4 through 10 further described below).

Again, referring primarily to FIG. 2, in particular embodiments, the one or more servers (2) can be used primarily as sources of computing device content, with primary responsibility for implementing the graphical user interface (57) being placed upon each of the one or more client or clinician computing devices (3)(4). As to these embodiments, each of the one or more client or clinician computing devices (3)(4) can download and run the appropriate portions of the application program (11) implementing the corresponding functions attributable to the client computing device (3) or clinician computing device (4) (as shown in the flow diagram of FIG. 3 as process (A) or (B) respectively).

The Graphical User Interface. Now referring primarily to FIGS. 2A, 2B, 2C through 10, the application program (11) in part includes a graphical user interface module (55) executable to generate a graphical user interface (57) which can be depicted on the display surface (59) of the client computing device (3) or the clinician computing device (4) which correspondingly allows a client user (108) or a clinician user (107) by user command (58) to execute one or more functions of the application program (11). The client user (108) or the clinician user (107) can interact with the graphical user interface (57) to execute one or more functions of the application program (11), which as illustrative examples, can include: selection of one or more control icon(s), entry of text into one or more fillable fields, voice command, keyboard stroke, mouse button point and click, touch on a touch screen, or otherwise, or combinations thereof (individually and collectively referred to as a “user command”).

The Signup Module. Now referring primarily to FIGS. 2A, 2B, 2C, 3 and 4, embodiments of the application program (11) can, but need not necessarily, include a signup module (60) which upon execution depicts a sign up menu (61) (as shown in the flow diagram of FIG. 3 as process (C)) which by user command (58) allows the client user (108) or the clinician user (107) to create an account (62) under which the client user (108) or the clinician user (107) can be authenticated by the system (1) and correspondingly receive authorization to access resources provided by or connected to the system (1) and access or load the application program (11).

The Login Module. Again, referring primarily to FIGS. 2A, 2B, 2C, 3 and 4, embodiments of the application program (11) can, but need not necessarily, include a login module (63) which upon execution depicts a login menu (64) which by user command (58) allows the client user (108) or the clinician user (107) to log in to an account (62)(as shown in the flow diagram of FIG. 3 as process (D)). To log in to an account (62), a user (107)(108) is typically required to authenticate oneself with a password (65) or other credentials, such as fingerprint or facial recognition, for the purposes of accounting, security, and resource management. Once the user (107)(108) has logged on, the system (1) will often use a user identifier (66) such as an integer to refer to them, rather than their username, through a process known as identity correlation. As one illustrative example, in UNIX® systems, the username (67) can be correlated with a user identifier (66).

The Setup Module. Now referring primarily to FIGS. 2A, 2B, 2C, 3 and 5 through 10, embodiments of the application program (11) can, but need not necessarily, include a setup module (68) which by user command (58) allows settings or values to be assigned to the application program (11) or the client computing device (3) or the clinician device (4)(as shown in the example of the flow diagram of FIG. 3 as process (E). Certain embodiments can, but need not necessarily, be provided with presets which provide settings or values automatically assigned by or to the application program (11) outside of user intervention. For the sake of brevity, the particular illustrative example depicted in FIGS. 5 through 9 and further described below, include only a client computing device (3); this not intended to preclude embodiments in which the operation of a clinician computing device (4) coordinates the operation of a plurality of client computing devices (3) in a computer administrator-computer user relationship within the system (1), or embodiments in which a client computing device (3) and a clinician computing device (4) have respective operation coordinated by the system (1) in which the client user (108) and the clinician user (107) have respectively a physician-patient or other relationships in which the program functions are discretely allocated between the clinician computing device (3) used by the client (108) and a clinician computing device (4) used by the clinician (107).

Now referring primarily to FIGS. 2A, 2B, 2C, 3 and 5 through 10, the setup module (68) can function to display a setup menu (69) on the display surface (61) of the client computing device (3) which, as an illustrative example, allows creation of a user profile (86) (shown in the example of the flow diagram of Figure as process (F)) by entry by user command (58) of one or more of:

age status (70), gender status (71), weight status (72), and height status (73) of the client user (50) that will interact with the assay device (12)(as shown in the example of FIG. 9). While the embodiment illustrated in FIGS. 5 through 9 respectively depict an age status field (74) in which age status values (75) (month, day, year) can be selected by user command (58), a gender status field (76) in which gender status values (77)(male, female) can be selected by user command (58), a weight status field (78) in which weight status values (79)(pounds or kilograms) can be select by user command (58), a height status field (80) in which height status values (81)(feet and inches or centimeters) can be selected by user command (58); this illustrative example is not intended to preclude depiction of additional or alternate status fields or status values relating to anatomical or physiological attributes of a client user (50), or medicaments (82) or food or drink (83) taken by a client user (50), including date and time (84)(85) in which medicaments (82) or food or drink (83) was taken by the client user (108).

Now referring primarily to FIG. 9, as to particular embodiments, the setup module (68) can further function to match the status values (75)(77)(79)(81) input by user command (58) into the set up menu (69) with the user identifier (66) to create a user profile (86) which can be stored remotely in the server memory (2) or locally in the client device non-transitory computer readable media (49).

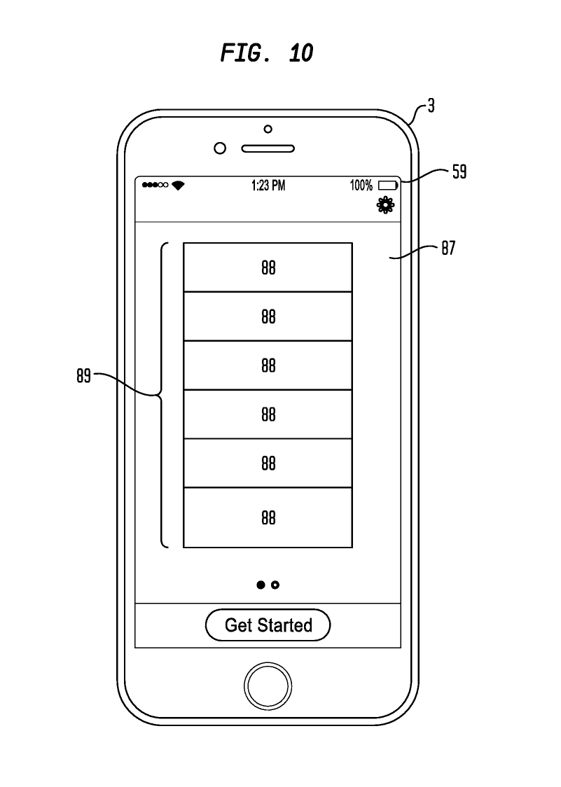

Now referring primarily to FIG. 10, as to particular embodiments, the setup module (68) can further function to depict on the display surface (59) of the client computing device (3) a condition selection menu (87) which allows by user command (58) selection of a condition (88) to be associated with the operation of an assay device (12) or the assay (45). As shown in the illustrative example of FIG. 10, the condition selection menu (87) depicts a conditions list (89) including a plurality of conditions (88) selectable by the client user (108).

The Communication Module. Now referring primarily to FIGS. 2A, 2B, 2C and 3, the application program (11) can further include a communication module (90) executable to communicate with an assay device (12). In particular embodiments, the client computing device (3) can be disposed in wired connection ( ) with the assay device (12), whether directly by crossover cable, or indirectly by wired connection through the network (5). In particular embodiments, the assay device (12) can be placed in the active condition by a client user (108) such that the assay device (12) seeks a connection (wired or wirelessly) with a client computing device (3) containing or having access to the application program (11). In particular embodiments, the communication module (90) can operate a radio frequency receiver (91) of the client or clinician computing device (3)(4) and the assay device (12) can correspondingly include a radio frequency controller (37) which operates a radio frequency transmitter (38) to transmit assay material reader pairing information (36) to cause wireless connection or pairing (shown in FIG. 3 as process (G)) of the client computing device (3) with the assay device (12) over a short-range radio frequency band (39) to carry a signal over all or a part of the communication path between the client computing device (3) and the assay device (12). The short-range frequency band (39) can include, as illustrative examples: BLUETOOTH® (40) which operates at frequencies of about 2402 MHz to about 2480 MHz or about 2400 MHz to about 243.5 MHz or WI-FI® (41) which operates at about 2.4 GHz or 5 GHz. In other particular embodiments, the assay device (22) can, but need not necessarily include, a tone generator (42) which generates tones (43) of the associated with an assay identification code (44) (also referred to as an “audio beacon”) that is linked to client computing device content (58) in the client computing device (3). In particular embodiments, the application program (11) or the communication module (90) can be further executed to record the date or time (84)(85) (also referred to as “a date and time stamp”) of pairing (A)(or with one or more subsequent communications or transfers of information or results) of the client computing device (3) with the assay device (12).

In particular embodiments, the assay device communication module (90) can, but need not necessarily, operatively communicate with the server (2) over the network (5) to communicatively couple the client computing device (3) with the assay device (12) and coordinate or pair operation of the client computing device (3) or the clinician device (4) with operation of assay device (21) as part of an internet of things (“TOT”) or TOT device (109). For the purposes of this invention the term “TOT device” means physical devices embedded with electronics, software, sensors, actuators and public, cellular, or other network connectivity to collect and exchange data and without reduction in the breadth of the foregoing definition includes as illustrative examples: glucose meters, blood pressure meters, intravenous pumps, activity or fitness monitors, or tooth brushes. In particular embodiments, the client computing device (3) and the assay device (22) can, but need not necessarily, be TOT devices part of the TOT.

The Global Positioning Module. Again, referring primarily to FIGS. 2A, 2B, 2C and 3, the client computing device (3) can further include a global positioning system receiver (9) which receives information from a global positioning system (10). The client computing device (3) or the clinician computing device (4) can further include a geographic location calculator (92) operable to calculate a geographic location (93) of the client computing device (3). The term “global positioning system (“GPS”)(10)”, for the purposes of this invention, means a plurality of earth-orbiting satellites (94) each transmitting a satellite positioning signal (95) continuously towards the Earth (96), that enables by operation of the geographic location calculator (92) determination of the geographic location (93) defined by location coordinates (97), including one or more of: a longitude (98), a latitude (99) or altitude (100) of the global positioning signal receiver (9) within the client computing device (3) (which may be shared with the clinician computing device (4)) or within the clinician computing device (4).

In particular embodiments, a global positioning module (101) of the application program (1) can be executed upon pairing (91) of the client computing device (3) with the assay device (12) to retrieve the location coordinates (97) of the client computing device (3) from the geographic location calculator (92). The location coordinates (97) can be further applied by the assay data processing module (15) as a global positioning reference (20b) in processing assay results (16) transmitted from the assay device (12) to the client computing device (3) to determine if the client computing device (3) has unlocked access (17) or locked access (18) to the assay results (16).

The Assay Data Processing Module. Again, referring primarily to FIGS. 2A, 2B, 2C and 3, in particular embodiments, the application program (11) can further include an assay table library (102) including a plurality of assay tables (103) each of which can be referenced by an assay identification code (44) associated with an assay (45). Upon pairing (91) of the client computing device (3) with the assay device (12), the assay data processing module (15) can receive the assay identification code (44) from the assay device (12) and correlate the assay identification code (44) transmitted by the assay device (12) with the corresponding assay table (103). An assay table (103) can include a plurality of data fields (104) each including or pointing to particular datum (105) associated with the assay identification code (44), or in which the datum (105) can be associated, with a particular assay identification code (44).

In particular embodiments, the assay data processing module (15) having associated the assay device identification code (44) with an assay table (103) in the assay table library (102) can, but need not necessarily, be further executed to depict instructions on the display surface (59) of the client computing device (3) regarding use of the assay material (22) with the assay material reader (24) (shown in the example of FIG. 3 as process (J)). In particular embodiments, the assay data processing module (15) can, but need not necessarily, be executed to depict an instruction on the display surface (59) of the client computing device (3) to dispose, associate or insert the assay material (22) into assay reader (24).

In particular embodiments, disposal or insertion of the assay material (22) into the assay reader (24)(shown in the example of FIG. 3 as process (K)) can, but need not necessarily, execute the reader assay data processor (31) to validate the assay material (22)(shown in the example of FIG. 3 as process (L)). For the purposes of this invention the term “validate” means to determine if the assay material (22) disposed or inserted in the assay reader (24) can be correlated with the assay identification code (44). If validation (I) results in a positive correlation, the assay reader (24) can further operate to run the assay (45) correlated with the assay identification code (44)(shown in Figure as process (M)), if validation (L) results in a negative correlation, the assay reader (24) can abort running the assay (45). Subsequent to performing the assay (45), the assay device (24) can transmit the assay results (16) to the paired client computing device (3)(shown in FIG. 3 as process (N)) (for off-line processing) or through the network (5) (for on-line processing) to the client computing device (3) (shown the example of FIG. 3 as process (O)).

In particular embodiments, the assay data processing module (15) can lock access (18) to the assay results (16) (shown in FIG. 3 as process (P)) or unlock access (17) to assay results (16) (shown in FIG. 3 as process (Q)) based on one or more references (19)(20a)(20b)(20c)(20d)(20e)(whether individually or in combination) correlated to one or more assay tables (103). For the purposes of this invention, the term “reference (19)” means a value or string of values that enables the assay data processing module (15) unlocked access (17) (shown the example of FIG. 3 as process (Q)) to the datum (105) in a data field(s)(104) contained in the accessed assay table (103) including the assay result (16).

In particular embodiments, if the assay results (16) associated with the assay identification code (44) received by the client computing device (3) cannot be correlated by the assay data processing module (15) by reference (19) to an assay table (103) contained in the assay table library (102), then the assay data processing module (15) can function to maintain the assay results (16) in the client computing device memory (49) with locked access (18) by the client computing device (3). Subsequently, the client computing device (3) can connect via the network (5) with the server (2) to download an updated assay table library (103) (process A). If the updated assay table library (102) can be correlated by reference (19) with the assay identification code (44), then the client assay data processing module (15) can, but need not necessarily, provide unlocked access (17) by the client computing device (3) to the assay table (103) and the corresponding assay results (16) and other assay datum (105).

In particular embodiments, upon pairing (process G) of the client device (3) with the assay device (12) the assay data processing module (15) can be executed to retrieve the geographic location (93) defined by location coordinates (97) determined by the geographic location calculator (92)(shown in FIG. 3 as process (R)). The data processing module (15) can be further executed to correlate a global positioning reference (20) with the assay table library (102). If the global positioning reference (20) correlates with an assay identification code (44), then the assay data processing module (15) can allow unlocked access (17) by the client computing device (3) to the assay table (103) and the corresponding assay results (16) and other assay datum (105). Conversely, if the retrieved location coordinates (97) associated with the client computing device (3) paired (A) with the assay device (12) cannot be referenced with an assay identification code (44) in an assay table (103) in the assay table library (102), the computing device assay data processing module (15) maintains locked access (18) by the client computing device (3) to the assay results (16) or other assay datum (105).

In particular embodiments, the data processing module (15) can be further executed to correlate a government or regulatory reference (20c) with each assay table (102) in the assay table library (103). The government or regulatory reference (20c) can reflect federal, state or local laws, regulations or rules (or similar laws, regulations, or rules of any foreign region, country or locality) which delimit the use of an assay device (12) or the release of the assay results (16) to the client computing device (3). As one illustrative example, the United States Food and Drug

Administration (“FDA”) maintains a medical device database of over-the-counter (“OTC”) medical devices and tests and (non-prescription devices and assays) that have been cleared or approved by the FDA, and also, maintains a medical device database of tests and medical devices that require a prescription from a clinician (107). If for example, the assay (45) has been cleared or approved by the FDA as an OTC assay device (12) for performing and OTC assay (45), the assay table (103) associated with the assay identification code (44) can be matched with the regulatory reference (20c) which allows the client computing device (3) access to the assay results (16) transmitted from the assay device (12). In particular embodiments, use of the assay device (12) an assay (45) may be delimited to a particular geographic locations (93). In the illustrative example, if upon pairing of the client computing device (3) with the assay device (12), the client device (3) has location coordinates (97) corresponding to a geographic location (93) in the United

States, then the global positioning reference (20b) can be further correlated with the assay identification code (44) and government or regulatory reference (20c) and the combination of references (19) allows the assay results (16) of the assay (45) to be accessed by the client computing device (3). Conversely, if the retrieved location coordinates (97) associated with the geographic location (93) of the client computing device (3) paired (process A) with the assay device (12) cannot be referenced with an assay identification code (44) in an assay table (103) in the assay table library (102) because the assay identification code (44) requires a clinician permission reference (20d), or a clinician access reference (20c), the computing device assay data processing module (15) maintains locked access (18) by the client computing device (3) to the assay results (16) or other assay datum (105).

In particular embodiments, the assay data processing module (15) can maintain locked access (17) to assay results (16) by the client device (3) unless the assay identification code (44) can be correlated with a clinician permission reference (20d). The assay data processing module (15) can be executed to receive clinician permission references (20d) which can be correspondingly associated with the assay identification code (44)(shown in FIG. 3 as process (S)). The clinician permission reference (20d), if correlated with the assay identification code (44) of one more assay tables (103) in the assay table library (102), can enable unlocked access (17) to the assay results (16) or other assay datum (105) contained in the referenced assay table (103) by the client computing device (3).

The assay data processing module (15) can be further executed to depict the assay result (16) on the display surface (59) of the client computing device (3)(shown in FIG. 3 as process (T)).

The Clinician Assay Data Processor. In particular embodiments, either the assay device (12) or the client computing device (3) can via the network (5) transmit assay test results (16) to the electronic medical record (52) associated with a client identification code (106) and which can include a date-time stamp (84)(85). A clinician (51) can access the electronic medical record (52) associated with the client identification code (106) and receive assay results (16) referenced to the client identification code (106). The clinician (51) can determine if the client computing device (3) should have unlocked access (17) to the assay results (16). If, yes, then the clinician (107) can by user command (58) in the clinician device (4) transmit a clinician permission reference (20d) to the client computing device (3). The clinician permission reference (20d) correlated with the client device identification reference (20a) and the assay identification code (44) contained in an assay table (103) within the assay table library (102) can enable unlocked access (17) by the client computing device (3) to the assay results (16) or other assay datum (105). The clinician permission reference (20d) can be transmitted via the network (5) and received by the client computing device (3).

As can be easily understood from the foregoing, the basic concepts of the present invention may be embodied in a variety of ways. The invention involves numerous and varied embodiments of a smart rapid result diagnostic system and methods for making and using such smart rapid result diagnostic system including the best mode.

As such, the particular embodiments or elements of the invention disclosed by the description or shown in the figures or tables accompanying this application are not intended to be limiting, but rather exemplary of the numerous and varied embodiments generically encompassed by the invention or equivalents encompassed with respect to any particular element thereof. In addition, the specific description of a single embodiment or element of the invention may not explicitly describe all embodiments or elements possible; many alternatives are implicitly disclosed by the description and figures.

It should be understood that each element of an apparatus or each step of a method may be described by an apparatus term or method term. Such terms can be substituted where desired to make explicit the implicitly broad coverage to which this invention is entitled. As but one example, it should be understood that all steps of a method may be disclosed as an action, a means for taking that action, or as an element which causes that action. Similarly, each element of an apparatus may be disclosed as the physical element or the action which that physical element facilitates. As but one example, the disclosure of an “assay” should be understood to encompass disclosure of the act of “assaying”—whether explicitly discussed or not—and, conversely, were there effectively disclosure of the act of “assaying”, such a disclosure should be understood to encompass disclosure of an “assay” and even a “means for assaying.” Such alternative terms for each element or step are to be understood to be explicitly included in the description.

In addition, as to each term used it should be understood that unless its utilization in this application is inconsistent with such interpretation, common dictionary definitions should be understood to be included in the description for each term as contained in the Random House Webster's Unabridged Dictionary, second edition, each definition hereby incorporated by reference.

All numeric values herein are assumed to be modified by the term “about”, whether or not explicitly indicated. For the purposes of the present invention, ranges may be expressed as from “about” one particular value to “about” another particular value. When such a range is expressed, another embodiment includes from the one particular value to the other particular value. The recitation of numerical ranges by endpoints includes all the numeric values subsumed within that range. A numerical range of one to five includes for example the numeric values 1, 1.5, 2, 2.75, 3, 3.80, 4, 5, and so forth. It will be further understood that the endpoints of each of the ranges are significant both in relation to the other endpoint, and independently of the other endpoint. When a value is expressed as an approximation by use of the antecedent “about,” it will be understood that the particular value forms another embodiment. The term “about” generally refers to a range of numeric values that one of skill in the art would consider equivalent to the recited numeric value or having the same function or result. Similarly, the antecedent “substantially” means largely, but not wholly, the same form, manner or degree and the particular element will have a range of configurations as a person of ordinary skill in the art would consider as having the same function or result. When a particular element is expressed as an approximation by use of the antecedent “substantially,” it will be understood that the particular element forms another embodiment.

Moreover, for the purposes of the present invention, the term “a” or “an” entity refers to one or more of that entity unless otherwise limited. As such, the terms “a” or “an”, “one or more” and “at least one” can be used interchangeably herein.

Thus, the applicant(s) should be understood to claim at least: i) the assay system or components thereof herein disclosed and described, ii) the related methods disclosed and described, iii) similar, equivalent, and even implicit variations of each of these devices and methods, iv) those alternative embodiments which accomplish each of the functions shown, disclosed, or described, v) those alternative designs and methods which accomplish each of the functions shown as are implicit to accomplish that which is disclosed and described, vi) each feature, component, and step shown as separate and independent inventions, vii) the applications enhanced by the various systems or components disclosed, viii) the resulting products produced by such systems or components, ix) methods and apparatuses substantially as described hereinbefore and with reference to any of the accompanying examples, x) the various combinations and permutations of each of the previous elements disclosed.

The background section of this patent application provides a statement of the field of endeavor to which the invention pertains. This section may also incorporate or contain paraphrasing of certain United States patents, patent applications, publications, or subject matter of the claimed invention useful in relating information, problems, or concerns about the state of technology to which the invention is drawn toward. It is not intended that any United States patent, patent application, publication, statement or other information cited or incorporated herein be interpreted, construed or deemed to be admitted as prior art with respect to the invention.

The claims set forth in this specification, if any, are hereby incorporated by reference as part of this description of the invention, and the applicant expressly reserves the right to use all of or a portion of such incorporated content of such claims as additional description to support any of or all of the claims or any element or component thereof, and the applicant further expressly reserves the right to move any portion of or all of the incorporated content of such claims or any element or component thereof from the description into the claims or vice-versa as necessary to define the matter for which protection is sought by this application or by any subsequent application or continuation, division, or continuation-in-part application thereof, or to obtain any benefit of, reduction in fees pursuant to, or to comply with the patent laws, rules, or regulations of any country or treaty, and such content incorporated by reference shall survive during the entire pendency of this application including any subsequent continuation, division, or continuation-in-part application thereof or any reissue or extension thereon.

Additionally, the claims set forth in this specification, if any, are further intended to describe the metes and bounds of a limited number of the preferred embodiments of the invention and are not to be construed as the broadest embodiment of the invention or a complete listing of embodiments of the invention that may be claimed. The applicant does not waive any right to develop further claims based upon the description set forth above as a part of any continuation, division, or continuation-in-part, or similar application.

1-15. (canceled)

16. A mobile computing device, comprising:
a processor communicatively coupled to a non-transitory computer readable media containing:
an electronic data exchanger operable to:
receive assay material reader pairing information from an assay material reader;
pair said mobile computing device with said assay material reader;
receive assay identification code from said assay material reader;
receive an assay result from said assay material reader;
an assay data processor operable to:
store said assay result; and
lock access or unlock access to said assay result by said mobile computing device.

17. The mobile computing device of claim 16, wherein said lock access or unlock access to said assay result further comprises a mobile computing device identification reference correlated with said assay identification code in an assay table, whereby correlation between said mobile computing device identification reference with said assay identification code in said assay table unlocks access to said assay result.

18. The mobile computing device of claim 17, further comprising a global positioning system receiver coupled to said mobile computing device to receive information from global positioning satellites to calculate a geographical location of said mobile computing device.

19. The mobile computing device of claim 18, wherein said lock access or unlock access to said assay result further comprises a global positioning reference correlated with said assay identification code in said assay table, whereby correlation between said mobile computing device reference and said assay identification code in said assay table unlocks access to said assay result.

20. The mobile computing device of claim 18, wherein said lock access or unlock access to said assay result further comprises a regulatory reference correlated with said assay identification code in said assay table, whereby a correlation between said regulatory reference and said assay identification code in said assay table unlocks access to said assay result.

21. The mobile computing device of claim 18, wherein said lock access or unlock access to said assay result further comprises a clinician access permission reference correlated with said assay identification code in said assay table, whereby a correlation between said clinician access permission reference and said assay identification code in said assay table unlocks access to said assay result.

22. The mobile computing device of claim 16, wherein said lock access or unlock access to said assay result further comprises a clinician access reference correlated with said assay identification code in said assay table, whereby a correlation between said clinician access reference and said assay identification code in said assay table unlocks access to said assay result.

23. The mobile computing device of any one of claim 16, wherein said mobile device assay data processor further operable to depict said assay result on a display surface of said mobile computing device.

24. The mobile computing device of claim 16, wherein said mobile device electronic data exchanger further operable to transmit said assay result to a medical records database, said assay result associated said mobile computing device identification reference, said assay result accessible by correlation with said clinician access reference.

25. The mobile computing device of claim 16, wherein said mobile device electronic data exchanger further operable to further receive a validation of correctness of said assay material received in said assay material detection zone of an assay reader.

26-43. (canceled)

44. A method in a mobile computing device, comprising:
pairing said mobile computing device with an assay material reader;
receiving in said mobile computing device an assay identification code from said assay material reader;
operating said assay material reader to read an assay material to detect a presence or amount of an analyte bound to said assay material;
receiving in said mobile communication device an assay result from said assay material reader;
operating a mobile device assay data processor, including:
storing said assay result in a non-transitory computer readable media of said mobile computing device;
correlating said assay identification code received from said assay material reader to an assay table accessible by said mobile computing device; and
locking access or unlocking access by said mobile computing device to said assay result.

45. The method of claim 44, wherein said locking access or unlocking access further comprises a mobile computing device identification reference correlated with said assay identification code in an assay table, whereby correlation between said mobile computing device identification reference with said assay identification code in said assay table unlocks access to said assay result.

46. The method of claim 45, further comprising receiving information from global positioning satellites to calculate a geographical location of said mobile computing device paired to said assay material reader.

47. The method of claim 44, wherein said locking access or unlocking access further comprises correlating a global positioning reference correlated with said assay identification code in said assay table, whereby correlation between said mobile computing device reference and said assay identification code in said assay table unlocks access to said assay result.

48. The method of claim 44, wherein said locking access or unlocking access further comprises correlating a regulatory reference with said assay identification code in said assay table, whereby a correlation between said regulatory reference and said assay identification code in said assay table unlocks access to said assay result.

49. The method of claim 46, wherein said locking access or unlocking access further comprises correlating a clinician access permission reference with said assay identification code in said assay table, whereby a correlation between said clinician access permission reference and said assay identification code in said assay table unlocks access to said assay result.

50. The method of claim 45, wherein said locking access or unlocking access further comprises correlating a clinician access reference with said assay identification code in said assay table, whereby a correlation between said clinician access reference and said assay identification code in said assay table unlocks access to said assay result.

51. The method of any one of claim 44, further comprising operating said mobile device assay data processor to depict said assay result on a display surface of said mobile computing device by operation of said.

53. The method of claim 45, further comprising transmitting said assay result to a medical records database, said assay result to a medical records database, said assay result associated said mobile computing device identification reference, said assay result accessible by correlation with said clinician access reference.

54. The method of claim 45, further comprising operating said reader assay data processor to compare an assay material identification data to said assay identification code to validate correctness of said assay material received in said assay material detection zone.

55-56. (canceled)
